(19) United States
(12) Patent Application Publication
(10) Pub. No.: US 2012/0136226 A1
(43) Pub. Date: May 31, 2012

(54) Pulse Oximetry For Determining Heart Rate Variability As A Measure Of Susceptibility To Stress

(75) Inventor: Thomas A. Wilke, Boulder, CO (US)

(73) Assignee: Nellcor Puritan Bennett LLC, Boulder, CO (US)

(21) Appl. No.: 12/955,187

(22) Filed: Nov. 29, 2010

(51) Int. Cl. A61B 5/0205

(52) U.S. Cl. 600/324

(57) ABSTRACT

Embodiments of the present disclosure relate to systems and methods for determining a physiologic parameter of a patient. Specifically, embodiments provided herein include methods and systems for determining or predicting the presence and/or severity of stress in a patient based on heart rate variability. The information relating to stress may be used as part of a broader physiological assessment.

### BACKGROUND

The present disclosure relates generally to a method and system for monitoring physiological parameters of a patient. Specifically, embodiments of the present disclosure relate to estimation of certain clinical parameters, such as susceptibility to stress, by determining heart rate variability through pulse oximetry measurements.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modem medicine.

One physiological parameter that physicians may wish to monitor is physiological stress. However, monitoring stress presents certain challenges. Stress is difficult to determine because its clinical manifestation often involves multiple and overlapping symptoms. Further, stress may involve both psychological and physiological components. In addition, a stress response may vary greatly between individuals.

Human subjects react to transient physiological stress in a variety of ways, including increased pulse rate, muscle reactions, circulatory changes, perspiration, and increased production of certain hormones. By monitoring the subject for these stress symptoms, the presence of the stress may be detected. For example, polygraph machines monitor pulse, respiration, and skin responses while the subject is interrogated. Unfortunately, polygraph machines provide only limited information about stress in certain controlled circumstances. Physicians may also monitor a patient for the presence of increased stress hormones, such as cortisol or norepinephrine. However, detection of these hormones is complex and time-consuming. Further, because baseline levels of these hormones may vary between individuals, analysis of concentration changes may be difficult.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor for determining heart rate variability in accordance with an embodiment;

FIG. 2 is a block diagram of an alternative patient monitor for determining heart rate variability in accordance with an embodiment;

FIG. 3 is a flow diagram of a method for determining heart rate variability in accordance with an embodiment;

FIG. 4 is a flow diagram of a time domain method that may be used in conjunction with the method of FIG. 3; and

FIG. 5 is a flow diagram of a frequency domain method that may be used in conjunction with the method of FIG. 3.

### DETAILED DESCRIPTION OF SPECIFIC EMBODIMENTS

One or more specific embodiments of the present techniques will be described below. In an effort to provide a concise description of these embodiments, not all features of an actual implementation are described in the specification, It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time-consuming, but would nevertheless be a routine undertaking of design, fabrication, and manufacture for those of ordinary skill having the benefit of this disclosure.

Heart rate variability is a measure of variation of heart rate (i.e., pulse rate) over time. Reduced heart rate variability has been used as a predictor for certain clinical conditions, such as mortality after myocardial infarction and the presence of coronary artery disease. A patient's heart rate variability on a beat-to-beat basis and within certain time windows has been shown to predict long term prognosis after cardiac events. While clinicians typically consider reduced heart rate variability to be associated with poorer prognoses, this association may be reversed for healthy and/or high-performing subjects. For these subjects, increased heart rate variability may be associated with and/or predictive of increased stress levels and, therefore, poorer performance, while reduced heart rate variability may be associated with lower stress and improved performance. For example, successful completion of military training exercises may be associated with reduced heart rate variability, either resting heart rate variability or a measured variability during the during the exercises. Accordingly, monitoring heart rate variability during activities that are considered stressful and/or taxing for a typical subject may allow observers to identify individuals who may be exceptional or high-performing (i.e., those with reduced heart rate variability) as well as individuals who may be likely to fail (i.e., those with increased heart rate variability). In addition, a patient's resting heart rate variability may be predictive of a patient's stress response, whereby a low resting heart rate variability is associated with the ability to withstand stress.

Heart rate variability may be monitored by an ECG or other time-sensitive device for providing heart rate data on a beat-to-beat basis with sufficient accuracy to detect small changes in the heart rate. However, ECG and other cardiac monitoring devices are unwieldy and require the proper placement of multiple electrodes that may interfere with a patient's mobility. As such, these devices may have limited usefulness in monitoring patients who are mobile and relatively active. As provided herein, a patient's stress level may be assessed by measuring heart rate variability via a patient monitor such as a pulse oximeter. Such monitoring may provide information to a clinician about a patient's physical condition. Such information may be used to direct therapy or to make decisions about a patient's fitness level. In particular cases, such information may be used to assess a patient's psychological stress levels.

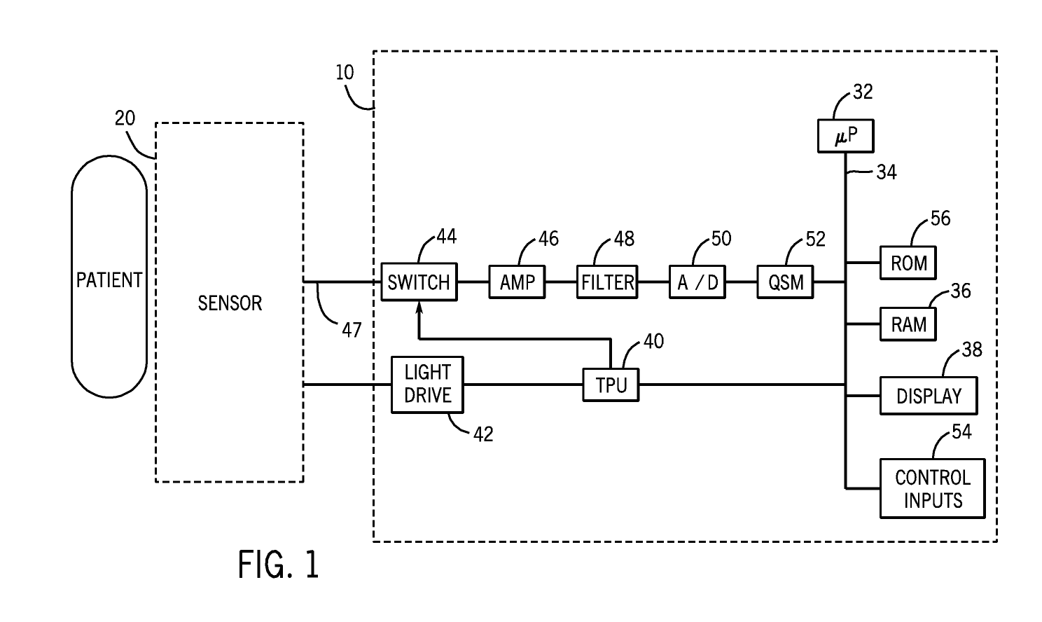

Heart rate variability may be assessed by any suitable patient monitoring system. For example, FIG. 1 is a block diagram of a patient monitor 10 that may be configured to implement the embodiments of the present disclosure. A pulse oximetry sensor 20 that includes optical components such as a light emitter (e.g., a light emitting diode) and a light detector (e.g., a photodetector) is applied to a patient and may be used to generate a plethysmographic waveform, which may be further processed by the monitor 10, The sensor 20 may be coupled to the monitor 10 wirelessly, which may be appropriate for heart rate variability monitoring during physical exercises, or via a cable. The monitor 10 may include a microprocessor 32 coupled to an internal bus 34. Also connected to the bus 34 may be a RAM memory 36 and a display 38.

A time processing unit (TPU) 40 may provide timing control signals to light drive circuitry 42, which controls when the optical components of the optical sensor (e.g., pulse oximetry sensor 20) is activated, and, if multiple light sources are used, the multiplexed timing for the different light sources. TPU 40 may also control the gating-in of signals from sensor 20 through a switching circuit 44. These signals are sampled at the proper time, depending at least in part upon which of multiple light sources is activated, if multiple light sources are used. The received signal from the pulse oximetry sensor 20 may be passed through an amplifier 46, a low pass filter 48, and an analog-to-digital converter 50. The digital data may then be stored in a queued serial module (QSM) 52, for later downloading to RAM 36 or ROM 56 as QSM 52 fills up.

Based at least in part upon the received signals corresponding to the light received by optical components of the pulse oximetry sensor 20, microprocessor 32 may calculate the oxygen saturation and/or heart rate using various algorithms, such as those employed by the Nellcor™ N-600x™ pulse oximetry monitor, which may be used in conjunction with various Nellcor™ pulse oximetry sensors, such as OxiMax™ sensors. In addition, the microprocessor 32 may calculate a heart rate variability using various methods, such as those provided herein. These algorithms may employ certain coefficients, which may be empirically determined, and may correspond to the wavelengths of light used. The algorithms and coefficients may be stored in a ROM 56 or other suitable computer-readable storage medium and accessed and operated according to microprocessor 32 instructions. In one embodiment, the correction coefficients may be provided as a lookup table.

Generally, heart rate variability may be determined from a plethysmographic waveform with relatively high resolution. A high resolution signal may capture smaller differences in beat-to-beat heart rate variability that may be masked by a lower resolution signal. The processor(s) 32 may determine the patient's physiological characteristics, such as SpO₂ and pulse rate, using various algorithms and/or look-up tables based generally on the value of the received signals corresponding to the light received by the detector. In certain embodiments, the processor(s) 32 may derive a desired physiological condition (e.g., arterial system compliance) based on one or more features (e.g., position of dicrotic notch) from received signals or a transformed versions (i.e., higher resolution) of the signals. For example, higher resolution signals may be obtained via continuous wavelet transformation as disclosed in U.S. application Ser. No. 12/437,317, entitled “Concatenated Scalograms,” filed May 7, 2009, and incorporated herein by reference in its entirety for all purposes. Embodiments of the present disclosure may utilize systems and methods such as those disclosed in U.S. application Ser. No. 12/437, 317, for obtaining information from the received signal to determine and to detect changes in physiological conditions.

In certain embodiments, the sampling rate for sampling of the analog signal 47 by the analog-to-digital converter 50 may be at least 2000 Hz. Accordingly, in one embodiment, the heart rate variability may be calculated from a signal that has been sampled at about 2000 Hz or higher. In embodiments in which the sampled signal is used for determining both blood oxygen saturation parameters as well as heart rate variability, the analog-to-digital converter 50 may either sample the entire signal at about 2000 Hz or higher or may sample the signal at higher rates only during time periods when the monitor 10 is collecting data for determining heart rate variability. For example, when a user provides an input to the monitor 10 to enable a heart rate variability monitoring function, a signal may be send to the analog-to-digital converter 50 to increase the sampling rate for the signal 47 during the time when a patient's heart rate variability is being monitored. In particular embodiments, the signal may be at least partially processed at the sensor 20. In such embodiments, the sensor 20 may include an integral analog-to-digital converter 50. After the heart rate variability has been determined and the user disables the heart rate variability monitoring function, the sampling rate may return to a lower resolution, e.g., to about 1200 Hz. Further, because increasing the sampling rate may also result in lower signal to noise ratios, the analog-to-digital converter 50 may be controlled at least in part by one or more signal quality metrics. For example, during periods when the signal is higher quality, the analog-to-digital converter 50 may increase the sampling rate during heart rate variability monitoring. During periods of lower signal quality, the analog-to-digital converter 50 may decrease the sampling rate during heart rate variability monitoring or may provide an indication that heart rate variability monitoring cannot occur.

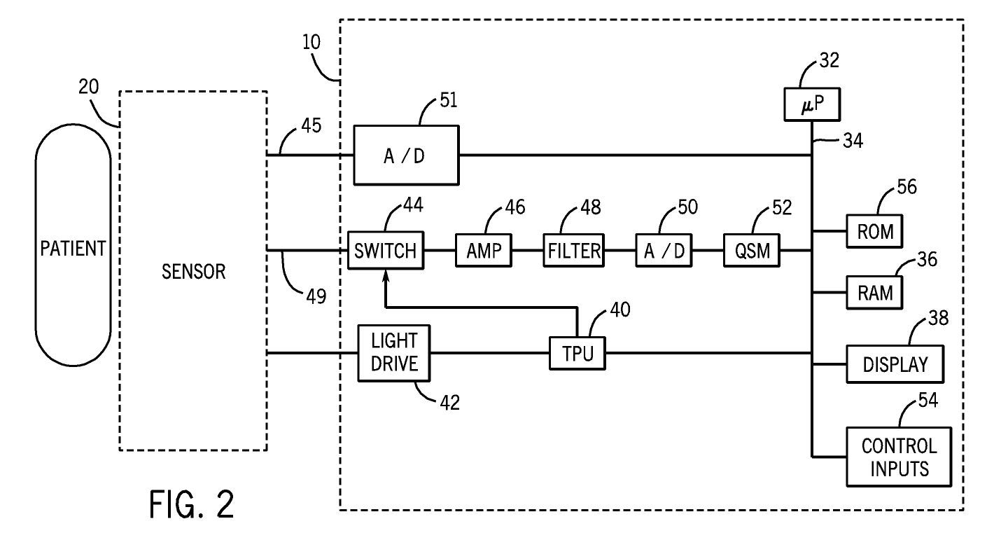

Certain types of signal processing may influence the ability of the microprocessor 32 to detect rapid changes in heart rate. Accordingly, in a particular embodiment, the pulse oximetry signal from the sensor 20 may be passed to the microprocessor 32 before any filtering has occurred (or may be passed to the microprocessor 32 after only minimal filtering, such as low pass filtering), as shown in the block diagram of FIG. 2. The signal 45 may be used to calculate the heart rate variability, while additional processing and filtering (e.g., amplifier 46, a low pass filter 48) may be applied the signal 49. The signal 45 may be sampled at a rate appropriate for calculating heart rate variability by a second analog-to-digital converter 51. Such a minimized signal processing arrangement prior to application of a heart rate variability calculation may facilitate detection of more rapid changes in beat-to-beat variation relative to a heavily processed signal, In such an embodiment, the heart rate for use in determining heart rate variability may be calculated separately (i.e., calculated from signal 45) from the heart rate used for display on the patient monitor (i.e., calculated from signal 49). In other embodiments, the algorithm for determining heart rate variability may use the heart rate as calculated for display at least in part for determining the heart rate variability.

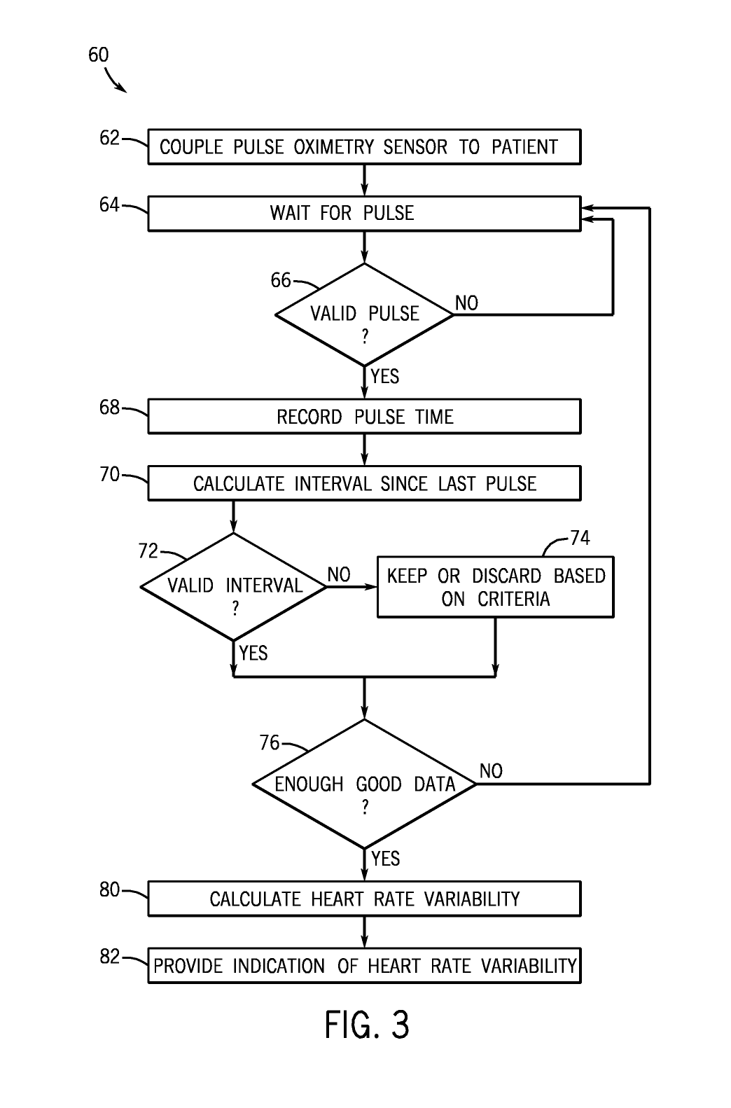

FIG. 3 is a process flow diagram illustrating a method 60 for determining heart rate variability in accordance with certain embodiments. The method may be performed as an automated procedure by a system, such as a system that includes a patient monitor 10 and a sensor 20. In addition, certain steps of the method may be performed by a processor, or a processor-based device such as a patient monitor 10 that includes instructions for implementing certain steps of the method 60. According to an embodiment, the method 60 begins with coupling a pulse oximetry sensor 20 to a patient at step 62 and waiting for a pulse signal from the sensor 20 at step 64. The monitor 20 may determine if a valid pulse has been generated at step 66 using any suitable signal quality assessment, such as those provided in U.S. Pat. Nos. 7,474,907, 7,039,538, and 6,035,223, the specifications of which are incorporated by reference in their entirety herein for all purposes. If the assessment indicates that a valid pulse has not been found, the method 60 returns to step 64 to continue to wait for a valid pulse. If a valid pulse has been obtained at step 64, the monitor 22 may record the time of the valid pulse (step 68) and calculate an interval since the last valid pulse (step 70).

The pulse interval validity may be determined by a rules-based method for determining allowable variation from historical and/or a calculated mean of a particular patient and by considering criteria such as artifact rejection, waveform smoothness, and noise between pulses. If the pulse interval is invalid, the method 60 may return to step 64 to wait for additional data. If the method 60 determines that a suitable pulse interval has been collected (step 72) over a suitable period of time (step 76), then a calculation of the heart rate variability may be performed (step 80).

After the heart rate variability has been determined, an indication of the heart rate variability may be provided to a caregiver (step 82). For example, the monitor 10 may provide a display or other indication to a clinician, such as a graphical, visual, or audio representation of the heart rate variability. For example, a heart rate variability associated with normal stress levels may include a numeric value or a green light indicated on a display or a short tone generated by a speaker associated with monitor 10. Similarly, a heart rate variability value associated with high stress may trigger an alarm, which may include one or more of an audio or visual alarm indication. In one embodiment, the alarm may be triggered if the heart rate variability value is substantially greater than a predetermined value or outside of a predetermined range. In one embodiment, the heart rate variability is expressed as a standard deviation from a beat-to-beat interval time. This value may be expressed as a raw numerical value (e.g., a time value), or may be provided as an index, for example by comparing the calculated variability to a threshold. In one embodiment, a heart rate variability value greater than 50 milliseconds (ms) or 75 ms may be considered to be indicative of a stressed individual or an individual who is prone to stress and/or who will have difficulty withstanding stress. Accordingly, depending on the threshold, the indicator may be scaled to a number of standard deviations from the cutoff, where a low index value represents a heart rate variability within one standard deviation of a threshold and a higher index value represents a heart rate variability that is more than one or two standard deviations from a threshold. The collected data may be over a particular time period, such as five minutes, or may involve a threshold of beat-to-beat data points, such as 300 or more beat-to-beat data points. Depending on the time window of the collected data, the threshold may be adjusted. For example, a longer collection time (e.g.,, greater than one hour) may be associated with a 70-100 ms cutoff (with less than 70-100 ms being associated with lower likelihood of stress), while a collection period on the order of around five minutes may be associated with a 30 ms cutoff (with less than 30 ms being associated with lower likelihood of stress).

In a particular embodiment, the heart rate variability may be expressed as a heart rate variability fraction. With a scatterplot of beat-to-beat data points divided into boxes (such as 256 boxes) of 0.1 second intervals, the two highest counts are divided by the total number of beats differing from the consecutive beat by <50 ms. The heart rate variability fraction may be obtained by subtracting this fraction from 1, and converting the result to a percentage

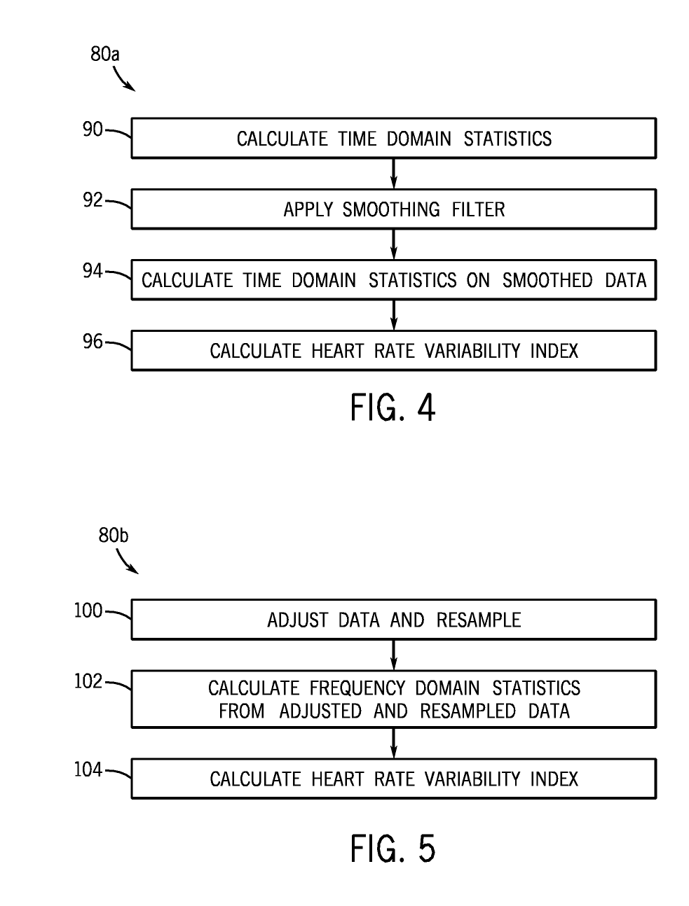

Determining heart rate variability at step 80 may be accomplished by any suitable method. Shown in FIG. 4 is an example of a time domain method 80a for determining heart rate variability. The heart rate variability may be determined at least in part by calculating time domain statistics at step 90 from the data collected from a pulse oximetry sensor, such as mean heart rate, standard deviation of pulse intervals (SDNN), square root of mean squared difference of successive pulse intervals (RMSSD), and the proportion of pulse intervals that differ from the mean (pNN50), After determining the time domain statistics, a smoothing filter, such as a finite impulse response filter, may be applied (step 92) to compensate for gradual shifts in pulse rate. In particular, calculating heart rate variability from a smoothed data set, in either the time or frequency domain, may improve the accuracy of the variability assessment, By smoothing data, any gradual changes in heart rate that may mask beat-to-beat variability may be eliminated. For example, if a patient's pulse rate increases over time as a result of increased activity, this gradual increase may mask beat-to-beat changes and, thus, may influence the heart rate variability. A smoothing filter may compensate for any gradual increase or decrease in heart rate. After smoothing, the time domain statistics may be calculated on the smoothed data (step 94).

A heart rate variability index or other metric for providing an indication to a caregiver (see FIG. 3, step 82) may be derived from one or more time domain statistics at step 96. For example, two or more statistical parameters may be combined. The statistical parameters may be the unsmoothed parameters or the smoothed parameters. In particular embodiments, a new parameter based on the difference between an unsmoothed and a smoothed parameter may be used. Confidence in the calculated index may be determined by a percentage of valid intervals in a particular data time window, or by a determination of signal quality.

In an alternative embodiment, heart rate variability at step 80 may be determined using frequency domain methods. Shown in FIG. 5 is an example of a frequency domain method 80b for determining heart rate variability. At step 100, the collected data may be adjusted for abnormal intervals. For example, any interval that falls outside of a predetermined range of interest (e.g., greater than 0.4 Hz) may be eliminated. Such ranges may be determined by a rules-based system based on historical limits and a calculated mean for a particular patient. In addition, the data may be resampled after certain abnormal intervals are removed to avoid introduction of artifacts.

At step 102, the adjusted and resampled data may be used to calculate frequency domain statistics. For example, a fast Fourier transform may be used to calculate a power spectral density, which is the magnitude of variability as a function of frequency. The power spectrum reflects the amplitude of the heart rate fluctuations present at different oscillation frequencies. The data may be divided into multiple frequency bands. For example, the spectrum may be divided into three or four different bands, depending on the major frequency bands. The boundaries of the frequency bands may be as follows: ultra low frequency <0,0033 Hz, very low frequency from 0.0033-0.04 Hz, low frequency from 0.04-0.15 Hz and high frequency from 0.15 to 0.4 Hz. A heart rate variability index or other metric may be calculated based on the frequency domain statistics at step 104. In one embodiment, the index may be based on one or more frequency domain statistical parameters. A confidence of the index may also be calculated, The confidence of the index may be related to the percentage of valid pulse intervals or other signal quality metric.

In addition to time domain and frequency domain methods, geometrical methods may be used to present pulse intervals in geometric patterns and to derive measures of heart rate variability. For example, a triangular index is a measure in which the length of the pulse interval serves as the x-axis of the plot and the number of each pulse interval length serves as the y-axis. The length of the base of the triangle is used and approximated by the main peak of the pulse interval frequency distribution diagram. Triangular interpolation approximates the pulse interval distribution by a linear function and the baseline width of this approximation triangle is used as a measure of the heart rate variability index, Alternatively, a Poincaré plot is another geometrical measure in which each pulse interval is plotted as a function of the previous pulse interval. Poincaré plots may be interpreted visually and also quantitatively.

In one embodiment, the method 60 may be used to determine a baseline, or resting, heart rate variability prior to a stressful event (e.g., a physical trial or a stresssful interaction, such as an interview or interrogation). The resting heart rate variability may be used as a predictor of a patient's response to the stressful event. High-performing subjects may have heart rate variabilities that considered to be low, e.g., within a standard deviation of a threshold. For subjects that may be considered low-performing or likely to experience stress, heart rate variability may be at least greater than a standard deviation, The threshold may be derived from an average heart rate variability calculated from a patient pool. For example, in one embodiment, a heart rate variability of 50 ms or less may be considered a normal heart rate variability. In other embodiments, a decreased percentage change from baseline relative to an empirically determined percentage change threshold (e.g., determined from an ideal subject or from a pool of subjects) may be used to determine whether a subject is stressed. If a subject has a particular heart rate variability at baseline and the heart rate variability decreases during the event, such a subject may be considered high-performing.

It is envisioned heart rate variability information may be useful to observers or clinicians in a variety of settings. A patient's heart rate variability may be monitored during an interview (e.g., an employment interview, a psychological assessment, or an interrogation) to determine stress levels. In such an embodiment, a subject may be monitored at a baseline or resting state prior to the interview. Monitoring may continue during a series of questions. Any increase in heart rate variability may be related to a stress response to the question being asked at the time. In such an embodiment, the monitor 10 may be configured to provide an indication of heart rate variability that is not easily interpreted by subject to avoid adding to the subject's stress. For example, the indication may be a visual marker on a display screen that is not visible to the subject. In other embodiments, the indication provided by the monitor 10 may be part of the stress assessment. For example, the monitor 10 may emit harsh tones or red light indicators upon increases in heart rate variability. If a subject is able to avoid further increases of heart rate variability under such conditions (e.g., exposure to physical stimuli from the monitor 10), the subject may be considered to have passed the interview.

In another embodiment, heart rate variability may be used during physical exercises as an indication of stress. Subjects that are stressed, i.e., that exhibit increased heart rate variability, may be more likely to panic or fail to complete the exercises. During complicated military training exercises, such an assessment may be used to cancel the exercises at convenient stopping points. For example, if a subject is stressed prior to performing ordinance-related exercises, the exercises may be halted before any ordinance is used. During diving exercises in which a subject may have limited ability to communicate with their handlers, stress monitoring may allow the subject to be pulled from the water before panic sets in and injury may occur. Heart rate variability monitoring may also be used to inform athletes of stressful portions of their performance. In one embodiment, a quarterback may be monitored for higher stress and lower stress plays. Lower stress may be associated with mastery of a particular play.

Heart rate variability may also be used as a predictive parameter for certain types of therapies and/or treatments. As noted, while reduced heart rate variability may be predictive of poorer prognoses in sick patients, reduced heart rate variability may be associated with lack of stress in healthier populations. Such patients may be able to withstand certain types of anesthesia or may be able to recover more rapidly from particular procedures. Accordingly, a physician may wish to determine a patient's baseline heart rate variability before prescribing general anesthesia or performing certain types of procedures.

Depending on the type of setting, a monitoring system may include instructions for monitoring heart rate variability that are specific to one or more scenarios. Such instructions may be stored on the monitor 10, on an associated multi-parameter monitoring system, on any suitable memory storage device, may be provided as a software update or add-on, or may be provided as written or graphical instructions. For example, for monitoring an athlete, the instructions may include instructions and settings for determining a baseline heart rate variability prior to exercise that include appropriate rest times for collecting baseline heart rate information. For monitoring subjects during an interview, the instructions may include instructions for facing a display away from the subject being interviewed so that the subject is not able to view any displayed changes in heart rate variability. It is contemplated that the instructions may be based on empirical results from previous monitoring studies or industry guidelines.

While the disclosure may be susceptible to various modifications and alternative forms, specific embodiments have been shown by way of example in the drawings and have been described in detail herein. However, it should be understood that the disclosure is not intended to be limited to the particular forms disclosed. Rather, the disclosure is to cover all modifications, equivalents and alternatives falling within the spirit and scope of the disclosure as defined by the following appended claims.

What is claimed is:

1. A method of determining relative stress level in a subject comprising:
using a monitor to:
receive a signal from a pulse oximetry sensor, wherein the signal is representative of a heart rate of a patient;
determine a heart rate variability of the subject based at least in part on the signal; and
determine or predicting a stress response of the subject based in part on the heart rate variability, wherein a higher determined or predicted stress response is associated with increased heart rate variability relative to a baseline heart rate variability.

2. The method of claim 1, wherein the signal is from a time period.

3. The method of claim 2, wherein the time period is associated with a stress event.

4. The method of claim 2, wherein the time period is associated with a physical activity, a psychological test, or a physical stimulus.

5. The method of claim 2, comprising using the monitor to sample the signal at a higher rate during a period when heart rate variability is being determined and sample the signal at a lower rate when heart rate variability is not being determined.

6. The method of claim 5, wherein using the monitor to determine the heart rate variability comprises assessing pulse intervals from the signal and rejecting invalid pulse intervals.

7. The method of claim 1, comprising using the monitor to provide an indication of stress based on the heart rate variability.

8. The method of claim 1, wherein using the monitor to determine the heart rate variability comprises applying a smoothing filter to the signal.

9. The method of claim 1, comprising using the monitor to receive a second signal from the pulse oximetry sensor, wherein the second signal is used to determine a blood oxygen saturation and a heart rate of the subject.

10. A monitor, comprising:
an input circuit configured to receive a pulse oximetry signal;
a memory storing an algorithm configured to calculate a heart rate variability based at least in part on the pulse oximetry signal;
a processor configured to execute the algorithm;
and
a display configured to provide an indication of stress based on the heart rate variability, wherein the indication of stress is associated with increased heart rate variability relative to a threshold.

11. The monitor of claim 10, wherein the algorithm comprises determining a pulse interval.

12. The monitor of claim 10, wherein the algorithm comprises a beat-to-beat heart rate variability determination.

13. The monitor of claim 10, wherein the algorithm comprises a time domain heart rate variability determination.

14. The monitor of claim 10, wherein the algorithm comprises a frequency domain heart rate variability determination.

15. The monitor of claim 10, wherein the algorithm comprises a geometric heart rate variability determination.

16. The monitor of claim 10, wherein the algorithm uses a signal quality input to determine a sampling rate for the pulse oximetry signal.

17. The monitor of claim 10, wherein the indication of stress comprises a numerical index or a graphical icon.

18. A system for determining patient stress, comprising:
a sensor configured to acquire pulse oximetry data and generate a signal relating to the pulse oximetry data; and
a monitor configured to:
sample the signal at a rate of 2000 Hz or higher;
determine a heart rate variability based at least in part on the signal; and
provide an indication of stress based on the heart rate variability.

19. The system of claim 18, wherein the indication of stress is associated with an increased heart rate variability as compared to a threshold or baseline value.

20. The system of claim 19, wherein heart rate variability below the threshold or baseline value is associated with a lack of stress.
